(19) United States
(12) Patent Application Publication
(10) Pub. No.: US 2005/0085746 A1
(43) Pub. Date: Apr. 21, 2005

(54) Retractable sheath introducer

(76) Inventors: Leland R. Adams, Ansonia, CT (US); Michael J. Bettuchi, Middletown, CT (US); Vincent J. Testa, Cranberry Township, PA (US)

Correspondence Address: FULWIDER PATTON LEE & UTECHT, LLP;HOWARD HUGHES CENTER, 6060 CENTER DRIVE, TENTH FLOOR, LOS ANGELES, CA, 90045, US

(21) Appl. No.: 10/688,398

(22) Filed: Oct. 16, 2003

(51) Int. Cl. A61M 25/00

(52) U.S. Cl. 600/585

(57) ABSTRACT

An introducer assembly for inserting a medical device into a body passage. The introducer assembly includes an elongate, tubular and retractable inner sheath formed of a deformable and flexible material. The introducer assembly further includes an inner core removably inserted through and secured to the inner sheath. A lumen within the inner core may serve as a channel for accommodating a guidewire. The introducer assembly is configured with a minimized outer diameter that avoids sheath kinking during insertion in the body passage while utilizing a thin flexible inner sheath capable of accommodating large-diameter catheters. A method for inserting a medical device through the introducer assembly is disclosed.

### BACKGROUND OF THE INVENTION

This invention relates to a medical device introducer, and more particularly to a flexible and retractable sheath sub-assembly and method for introducing a catheter or other medical device into a patient's vasculature.

Introducers are used to assist in the introduction of medical devices, such as catheters, into a patient's vascular system. The introducer must penetrate the skin and wall of a blood vessel or other body passage and be positioned within the lumen of the vessel so that a medical device can be advanced and withdrawn through the introducer. Introducer assemblies are typically configured with an elongated tubular member open at both ends, so as to form a sheath, a tubular dilator or other core member slidably positionable within the sheath, and a guidewire slidably positionable within the dilator.

Catheters may be introduced from the exterior of a patient into a body passage, such as an artery or vein. After introduction, the catheter may be advanced through the patient's vasculature to a desired site. The catheter may be so placed by first inserting an introducer needle or other device through the skin into a desired lumen. A guidewire may then be passed through the needle and advanced within the body passage toward the desired site for positioning the medical device. The needle is then withdrawn, leaving the guidewire in the passage. A catheter introducer may then be placed over the guidewire and further advanced into the body passage. The distal end of the catheter may be then advanced through the introducer. Once the distal end of the catheter is positioned at the desired location within the patient's vasculature, the guidewire and introducer may be removed from the body passage.

The outer diameter or profile of an introducer assembly should be minimized to reduce the obstruction of blood flow in the blood vessel when the introducer is inserted into the body passage and to limit the size of the incision required to insert the introducer over the guidewire. A minimized introducer outer diameter aids in reducing the trauma suffered by the vessel wall, thus shortening the patient's recovery time. Accordingly, a small-diameter introducer sheath may be used; however, a drawback to such use is that large-diameter catheters cannot be accommodated. An alternative implementation is to use a large-diameter introducer sheath characterized by a thin wall to reduce the profile of the sheath; however, this type of sheath may be subject to damage that would result in a reduction in annular space (e.g., kinking) during the insertion process.

A known problem with prior art introducers is that a thin sheath may be drawn away from intimate engagement with the dilator when the introducer is advanced within the body passage. This occurs when the sheath material is forced outwardly from its close-fitting relation with the dilator because the plastic material of the thin, tubular sheath lacks the hoop strength to avoid slight, undesired radial stretching at the distal tip during insertion of the sheath. Attempts to solve this problem by using sheath material with high hoop strength have been unsuccessful, since such a sheath is typically unduly stiff for traversing tortuous anatomy.

Therefore, there is a need for an introducer assembly having a minimized outer diameter that avoids sheath kinking during insertion into a body passage while utilizing a retractable, flexible sheath capable of accommodating large-diameter catheters.

### SUMMARY OF THE INVENTION

The present invention is directed to an introducer assembly for inserting and guiding a medical device (instrument), such as a catheter assembly, into a body passage. In particular, the introducer assembly includes a retractable, elongate, tubular inner sheath formed of a flexible material. A steerable, torqueable, elongate, tubular outer sheath is disposed over the inner sheath, and a relatively stiff inner core configured to accept a guidewire is disposed within and may be secured to the inner sheath. The proximal end of the introducer assembly may include a handle or other assembly to allow the user to manipulate the distal end of the introducer assembly. The distal end of the introducer assembly may be tapered to facilitate insertion into the body passage, such as the femoral vein of a human patient.

The introducer assembly of the present invention may include an inner core or dilator removably positioned within the inner sheath. The inner core may be configured from a solid cylindrical annular element having an inner diameter and an outer diameter. A passage through the inner diameter of the inner core may serve as a channel or lumen for accommodating the guidewire. The distal tip (end) of the inner core may be provided with a forwardly descending conical tapered surface. Toward the distal section, proximal the conical tapered surface, the inner core may have a narrowed portion for accommodating a release mechanism (e.g., a wire or cord) for removably securing the distal portion (end, section) of the inner sheath to the distal portion (end, section) of the inner core. The distal portion of the inner sheath may be folded or otherwise manipulated to reduce the outer diameter (profile) of the introducer during insertion into the body passage.

The inner sheath and inner core are configured to be removably inserted through the outer sheath, which may be configured with an inner diameter smaller than an outer diameter of the expanded inner sheath. In addition, the outer sheath of the introducer assembly is preferably formed from a material flexible enough to provide ease of insertion and tracking, yet stiff enough to advance through resistance encountered in the vasculature (i.e., resist kinking) when traversing through tortuous anatomy. By housing the inner sheath, the outer sheath serves to protect the inner sheath from kinking during insertion of the introducer assembly in the body passage. When the outer sheath is configured with an inner diameter smaller than the outer diameter of the inner sheath, the inner sheath is removably inserted through the outer sheath by radially folding the inner sheath to fit within the inner diameter of the outer sheath. Therefore, the outer sheath also serves to keep the inner sheath in a folded state. Further, by utilizing the outer sheath with a smaller diameter, the profile of the entire introducer assembly is reduced, thus reducing the obstruction of blood flow and patient trauma when the introducer assembly is inserted in the body passage.

An introducer assembly of the present invention is configured to introduce a medical device or instrument into a vasculature of a patient, includes a first sheath formed from a retractable material, and includes a second sheath formed from a flexible material. The introducer further includes a core member having a longitudinal lumen, and includes a ripcord having a distal section wound about, tied to or otherwise configured to removably attach the first sheath to the core member. When assembled, the core member is disposed within the first sheath, which is disposed within the second sheath. The proximal section of the ripcord may be disposed between the first sheath and the second sheath, or may be disposed within the lumen of and secured to the core member. The introducer may also include a guidewire disposed with the lumen of the core member having a tapered distal section. In addition, the first sheath may be configured with a handle adapted to receive the guidewire, and the lumen of the core member also may be adapted to receive the guidewire.

The present invention also contemplates a method for introducing a medical instrument into the vasculature of a patient. The method includes providing an introducer assembly of the present invention, for example, an introducer having an inner core, an inner sheath, a release mechanism and an outer sheath. A guidewire is inserted into a vasculature of a patient and the introducer assembly is threaded or otherwise moved over the guidewire and into the vasculature of a patient. When the distal portion of the introducer assembly is at a desired site within the patient, the outer sheath is withdrawn from the vasculature. The release mechanism is used to free the distal portion of the inner sheath from the distal portion of the inner core, for example, by moving the distal portion of the inner core, or by moving the distal portion of the inner sheath. The inner core and release mechanism are then sequentially or simultaneously withdrawn from the vasculature. The medical instrument is then inserted into the proximal portion of the inner sheath and the medical instrument is advanced within the inner sheath and into the vasculature. The inner sheath may then be retracted from the vasculature.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 depicts a plan view of an introducer assembly of the present invention.

FIG. 2 depicts a plan view of an introducer assembly of the present invention, wherein the outer sheath has been removed from the assembly.

FIG. 3 depicts a plan view of an introducer assembly of the present invention, wherein the outer sheath and release mechanism have been removed from the assembly.

FIG. 4 depicts a plan view of the inner sheath and handle of an introducer assembly of the present invention.

FIG. 5 depicts a plan view of an inner core of an introducer assembly of the present invention.

FIG. 6 depicts a plan view in partial cross-section of an introducer assembly of the present invention.

FIGS. 6a, 6b, 6c depict alternate embodiments of a folded inner sheath of the present invention taken along the line 6a-6a of FIG. 6.

FIG. 7 depicts a plan view in partial cross-section of an inner sheath of the present invention, wherein the sheath has been retracted from a patient's vasculature.

FIG. 8 depicts a plan view of an alternative embodiment of the inner core of an introducer assembly of the present invention, wherein the inner core is configured to secure a release mechanism.

FIG. 9 depicts a plan view of the inner sheath and handle of an introducer assembly of the present invention, wherein the inner sheath is disposed over the inner core, which contains a release mechanism.

FIG. 10 depicts a plan view in partial cross-section of the distal end of a guidewire positioned within a body passage.

FIG. 11 depicts a plan view in partial cross-section of an introducer assembly of the present invention positioned within a body passage.

FIG. 12 depicts a plan view in partial cross-section of an outer sheath of the present invention being withdrawn from a body passage.

FIG. 13 depicts a plan view in partial cross-section of an inner core and a ripcord of the present invention being withdrawn from a body passage.

FIG. 14 depicts a plan view in partial cross-section of a guidewire being withdrawn from the distal end of an inner sheath of the present invention positioned within a body passage.

FIG. 15 depicts a plan view in cross-section of a dilator being inserted into a proximal opening of an inner sheath of the present invention.

FIG. 16 depicts a plan view in partial cross-section of a medical device being advanced through a body passage within an inner sheath of the present invention.

FIG. 17 depicts a plan view in partial cross-section of an inner sheath of the present invention having been partially withdrawn from the distal end of a medical device positioned within a body passage.

### DETAILED DESCRIPTION OF THE PREFERRED EMBODIMENTS

In general, the introducer assembly of the present invention is configured for inserting a medical device or instrument, such as a catheter, into the body of a patient, e.g., into the patient's vasculature. The introducer assembly includes an elongate, tubular, retractable inner sheath formed of a flexible material for inserting and guiding a medical device within a body passage and for protecting the patient's vasculature. The introducer assembly further includes a dilator or inner core removably inserted through the inner sheath for receiving a guidewire. The inner core is defined by a solid cylindrical annular element having an inner diameter and an outer diameter. A passage or lumen through the inner diameter of the inner core may serve as a channel for slidably receiving a guidewire. Further, a releasing mechanism may be provided to removably affix the distal portion of the inner sheath to the distal tip of the inner core, for example by tying the inner sheath to the inner core with a string, suture, wire or similar filament (ripcord). The inner core may have a narrowed portion near the distal tip to facilitate attachment of the releasing mechanism.

The introducer assembly further includes an elongate tubular outer sheath formed of a flexible material formed to provide the necessary steerability, torqueability and trackability to traverse human vasculature for deployment of a medical device into the superior vena cava of a human patient. The outer sheath protects the inner sheath during insertion, and maintains the inner sheath in a folded state. The outer sheath is further configured to maintain a reduced introducer assembly profile when the inner sheath and the inner core are positioned within the outer sheath, and to maintain the annular space within the inner sheath (i.e., resist kinking) during insertion of the introducer assembly into the body of the patient.

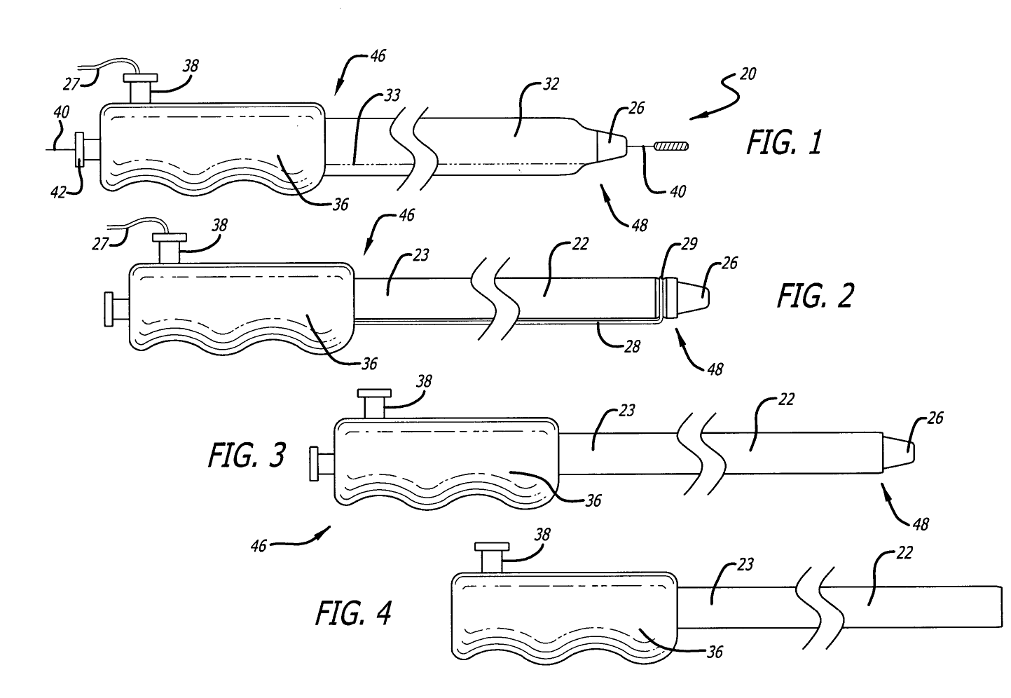

Referring now to the drawings for purposes of illustration and particularly to FIGS. 1-9, the introducer assembly 20 is configured to introduce large-diameter catheters and other medical devices into a body passage. The introducer assembly includes several elongate tubular sub-assemblies nestled concentrically within each other. The outermost member of the introducer assembly is a generally cylindrical outer sheath 32, which extends form the handle 36 positioned at the proximal end 46 of the introducer assembly to the distal portion (end, section) 48 of the introducer assembly (FIG. 1). The outer sheath is preferably formed from a material that is highly flexible, yet contains enough hoop strength to avoid kinking of the introducer assembly during insertion into the body passageway. The distal portion (end, section) of the outer sheath may be tapered or otherwise contoured to reduce the entry profile of the introducer assembly and to minimize trauma to the body passageway during insertion of the introducer assembly.

Accordingly, the outer sheath 32 may have a wall thickness in the range of about 0.005 to 0.025 inches (0.013 to 0.064 centimeters), preferably approximately 0.01 inches (0.025 cm), and be formed of a number of suitable materials, such as polytetrafluoroethylene (PTFE), polyimide or pebax. The outer diameter of the tubular outer sheath is configured smaller than the inner diameter of the target vessel, while still having a large enough diameter to accommodate the rest of the sheath sub-assemblies in their insertion configuration. The circumference and/or length of the outer sheath may be formed with perforations 33 or other mechanisms for facilitating removal of the outer sheath from the other sheath sub-assemblies.

The introducer assembly 20 further includes a flexible, retractable tubular elongate inner sheath 22 having a passage (lumen) extending from its proximal portion (end, section) to its distal portion (end, section) so as to accommodate a medical device, such as an intravascular catheter. The proximal portion of the inner sheath may be fixedly or removably secured to a handle 36 of the introducer assembly (FIG. 4). The length of the inner sheath extends from outside of the insertion site (incision) to the location in the body passage where the medical device is desirably positioned. So as to facilitate flexibility and retract-ability, the inner sheath may be formed with a wall thickness in the range of about 0.002 to 0.1 inches (0.005 to 0.25 cm), preferably approximately 0.005 inches (0.013 cm). Suitable flexible, retractable and expandable material for forming the inner sheath includes, but is not limited to, polytetrafluoroethylene (PTFE).

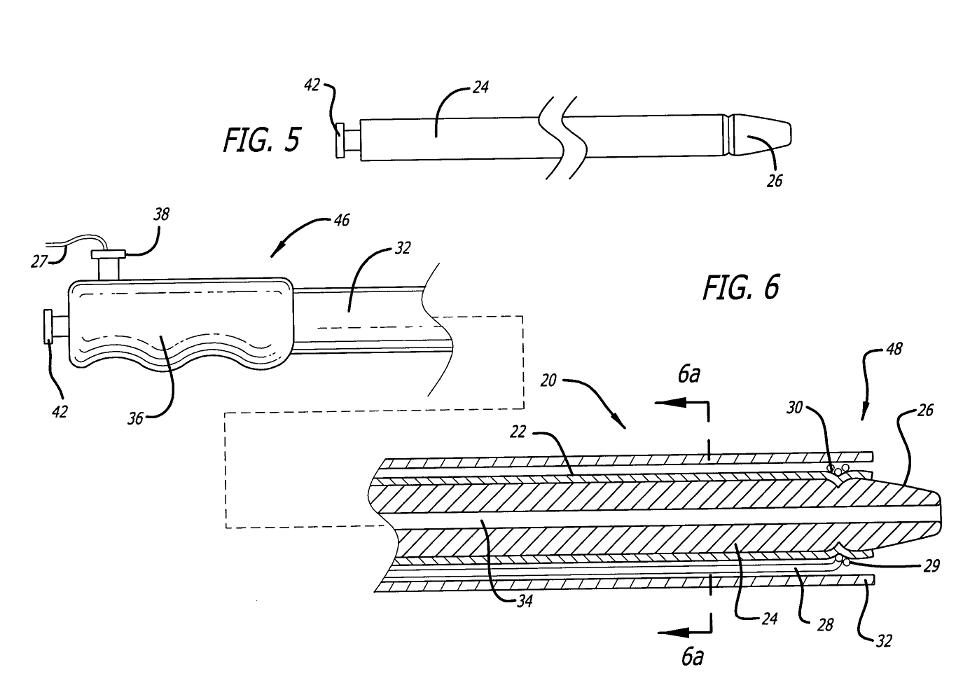

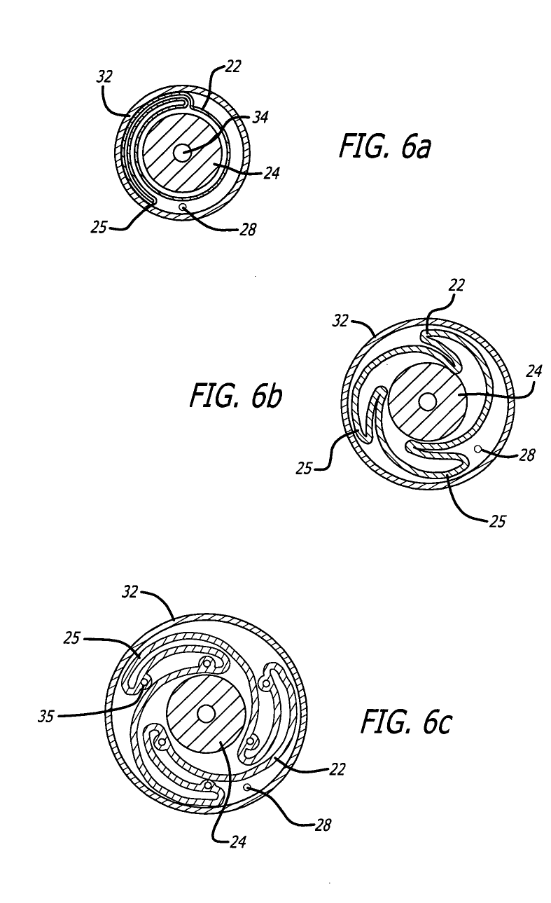

The introducer assembly 20 further includes a dilator or inner core 24 removably inserted through the inner sheath 22 for receiving an introduction guidewire (FIGS. 5, 6). The inner core may be formed from a solid cylindrical annular element having an inner diameter and an outer diameter. The inner core may have a length somewhat longer than the inner sheath. A passage through the inner diameter of the inner core forms a guidewire channel 34 and is sized such that it may accommodate a guidewire with a diameter of about 0.038 inches (0.097 cm). The inner core may further have a distal tip (portion, end, section) 26 characterized by a forwardly descending conical taper. Such a conical tip is useful during insertion for guiding and sliding the assembly along and through a body passageway. In addition, the inner core may be used to expand the inner sheath when the inner sheath is in a wrapped insertion configuration (FIGS. 6a, b, c).

The introducer assembly 20 also includes a releasing mechanism (such as a ripcord) 28 that may be configured for removably fastening a distal portion (end, section) of the inner sheath 22 proximate the distal tip 26 of the inner core 24 (FIG. 2). The ripcord may have a similar length comparable to the inner core so that the proximal end of the ripcord may be manipulated from proximate the handle 36 on the proximal end 46 of the introducer assembly. A segment of the inner core located proximal from where its distal tip (end, section) descends into a conical taper may include a narrowed portion or neck 30 to assist in securing the release mechanism to the inner sheath (e.g., tying the ripcord), and thereby to the inner core (FIGS. 2, 5, 6). The ripcord may be configured such that a pull by the user (physician) on the proximal portion 27 of the ripcord will unfasten a knot or windings 29 that tie or otherwise removably affix the inner sheath to the inner core. In addition, the releasing mechanism may be configured and positioned so as to aid in removal of the outer sheath 32. For example, the filament or wire may be of a diameter to open an adjacently positioned perforation, indentation or other alteration 33 of the sheath wall to aid in cutting or tearing the outer sheath material (FIG. 1).

Preferably, the inner sheath 22 having the inner core 24 disposed within its lumen (FIG. 3) is removably inserted through or otherwise positioned within the lumen of the outer sheath 32 (FIG. 6). Accordingly, the introducer assembly 20 has an overall length that permits placing the distal end 48 of the assembly proximate the desired location for positioning the medical device 50 within a patient vasculature or other body passageway 45, e.g., the superior vena cava. The outer sheath 32 may have a length substantially the same as the inner sheath 22, or the inner sheath may be shorter or longer than the outer sheath, as dictated by the particulars of traversing the medical device to the target anatomy. As shown in FIG. 1, the distal end of the outer sheath may be tapered and extend distal from the distal end of the inner sheath so as to reduce the number of exposed distal edges when introducing the assembly through the patient vasculature.

So as to minimize the overall outer diameter or profile of the introducer assembly 20, the material of the inner sheath 22 is preferably folded upon itself forming one or more leafs 25, as shown in FIGS. 6a, 6b, 6c. Positioning the folded inner sheath within the outer sheath 32 serves to reduce the insertion profile of the inner sheath, thus helping to minimize the profile of the entire introducer assembly. Folding the inner sheath further serves to protect the inner sheath from damage (e.g., resist kinking) when disposed within the stiffer (higher radial strength) tubular outer sheath. Similarly, the outer sheath protects the inner sheath from undesired radial stretching at its distal end during insertion of the assembly into the body passageway. The inner sheath may also include a mechanism for self-expansion 35 (such as ridges or imbedded memory-shape wires) when the outer sheath is retracted from the inner sheath (FIG. 6c).

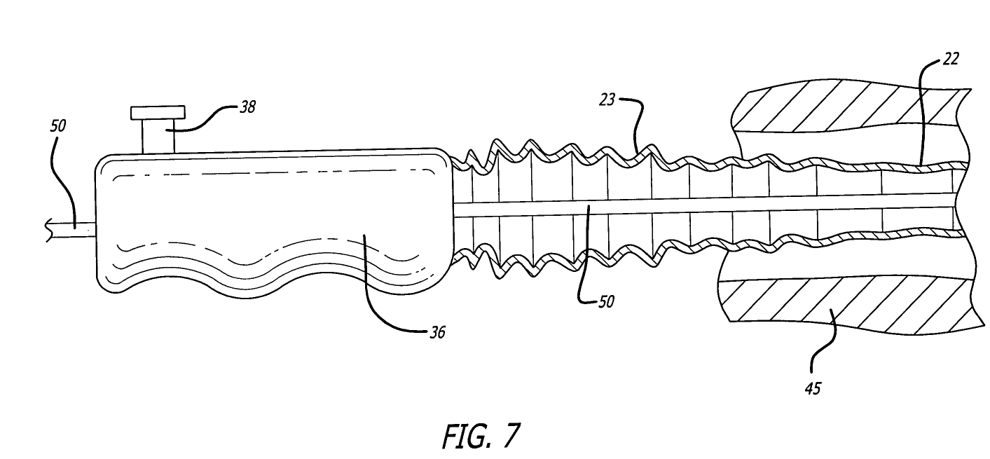

As shown in FIG. 7, the inner sheath may be formed of a material that is flexible enough so as to be retracted over the medical device 50 from outside of the patient's body. The inner sheath's proximal end may be removed from the handle 36 prior to retraction of the inner sheath from the patient. Likewise, the inner sheath may be detached from the handle prior to insertion of the medical device, wherein a separate dilator may be employed to open the proximal end of the inner sheath (FIG. 15).

When configuring the introducer assembly for introducing a catheter proximate a human vena cava, the introducer assembly profile is preferably of a size of about twenty French (0.6 cm); whereas the outer diameter of the expanded inner sheath is preferably about thirty-four French (1.02 cm). Such a reduced overall delivery profile assists in avoiding trauma to the vessel wall during insertion. Further, obstruction of blood flow is reduced during insertion of the medical devices, thereby shortening patient recovery time.

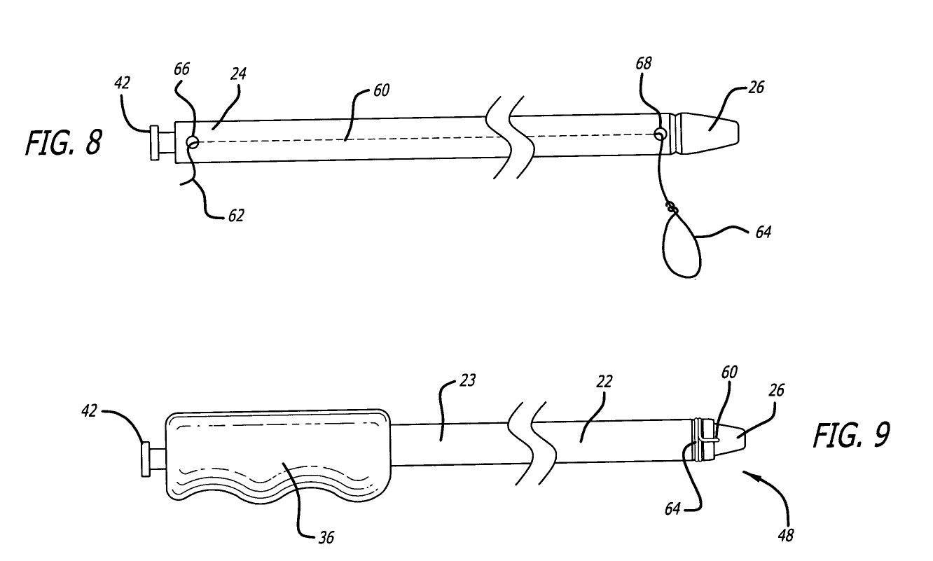

As shown in FIGS. 8 and 9, an alternate embodiment the release mechanism 28 of the present invention may include a suture, wire, ripcord or similar device 60 that is at least partially disposed within the inner core 24. For example, a suture may be threaded or otherwise positioned within the inner core's lumen (guidewire channel) 34 (FIG. 6a). The proximal end 62 of the suture may secured to the inner core at a point distal of the guidewire exit port 42, for example, at a slit or port 66 in the wall of the inner core (FIG. 8). The distal end 64 of the suture may exit the inner core lumen proximal the inner core's distal end 26, for example, at a slit or port 68 in the wall of the inner core near its tapered tip. The distal end of the suture may be wrapped around, tied in a loop, permanent knot or otherwise secured to the distal portion of the inner sheath 22 (FIG. 9). The outer sheath is placed over the inner sheath and inner core. As described herein, the distal end of the introducer assembly 48 is advanced into position within the patient's vasculature, and the outer sheath is removed from the vasculature. The inner core may be then pushed forward slightly to release the distal end of the suture from the inner sheath. Thereafter, the inner core and suture are together retracted through the inner sheath and removed from the patient's vasculature.

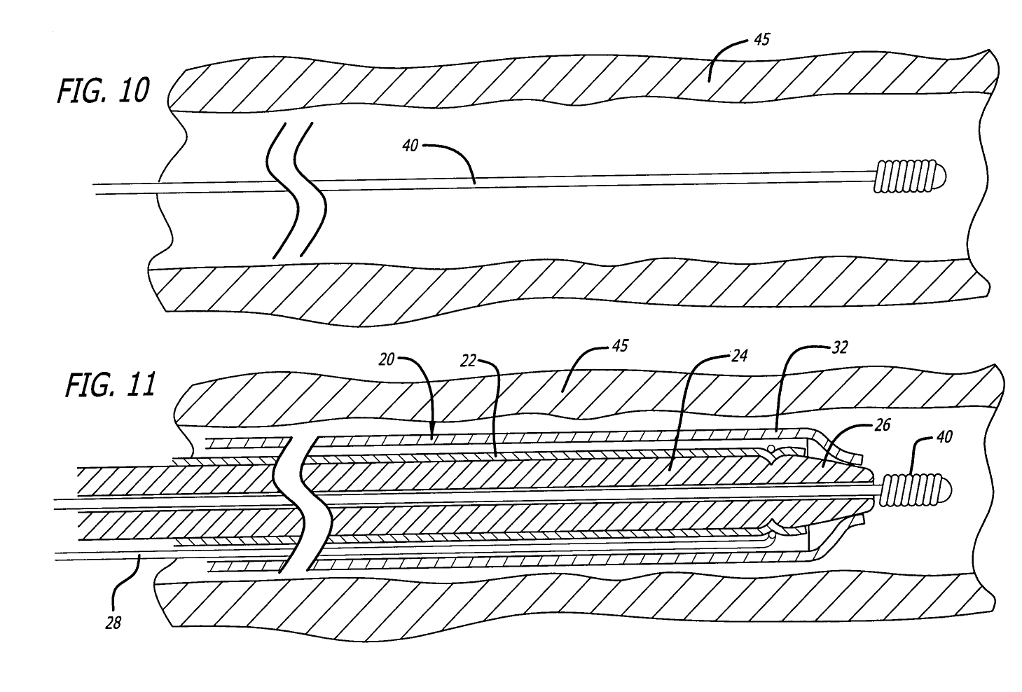

Referring now to FIGS. 10-17, the method of the present invention initially involves providing an introducer assembly 20 and medical device 50, and then identifying an insertion site, such as for access to a femoral vein, on the body of a patient. An introducer needle (not shown) is inserted into the site, after which the needle may then be manipulated until the needle tip is positioned near the center of a body passage. A syringe (e.g., 10 ml) may be attached to the needle (e.g., 18-gage) so that blood may be drawn into the syringe to assure proper placement in an artery or vein. Thereafter, the syringe may be detached from the needle. A guidewire 40 may be inserted into the needle and fed into the vein until the guidewire distal tip reaches a desired location in the patient. The introducer needle may be then removed from the insertion site so as to leave the guidewire in the vessel, as shown in FIG. 10. Alternatively, the introducer assembly may be inserted directly into a incision and into the target vessel(s).

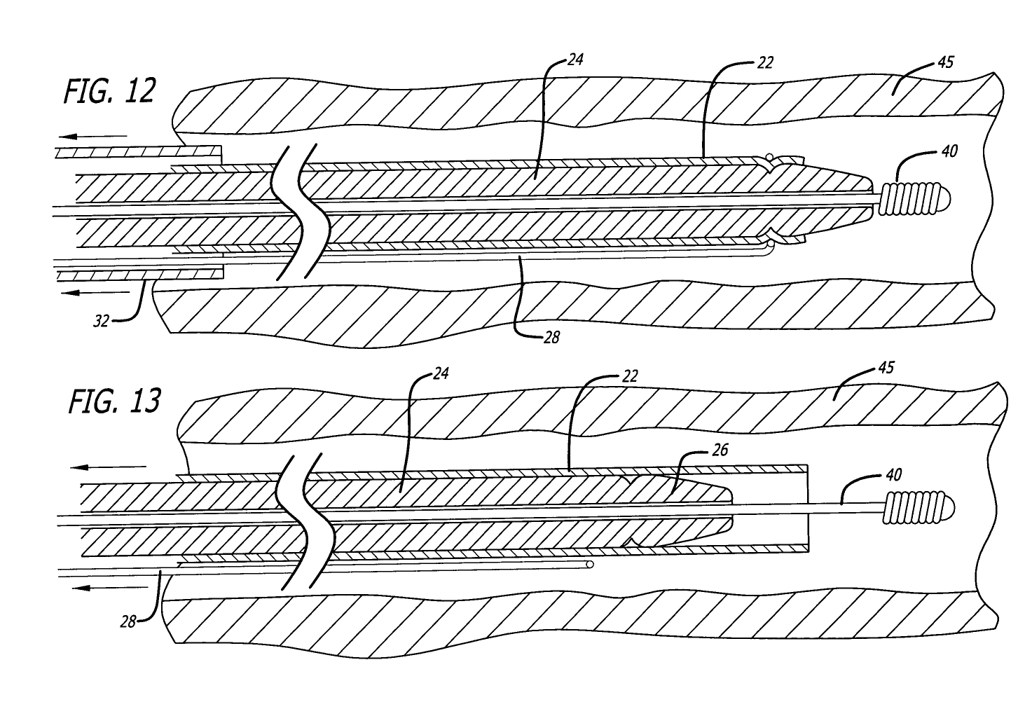

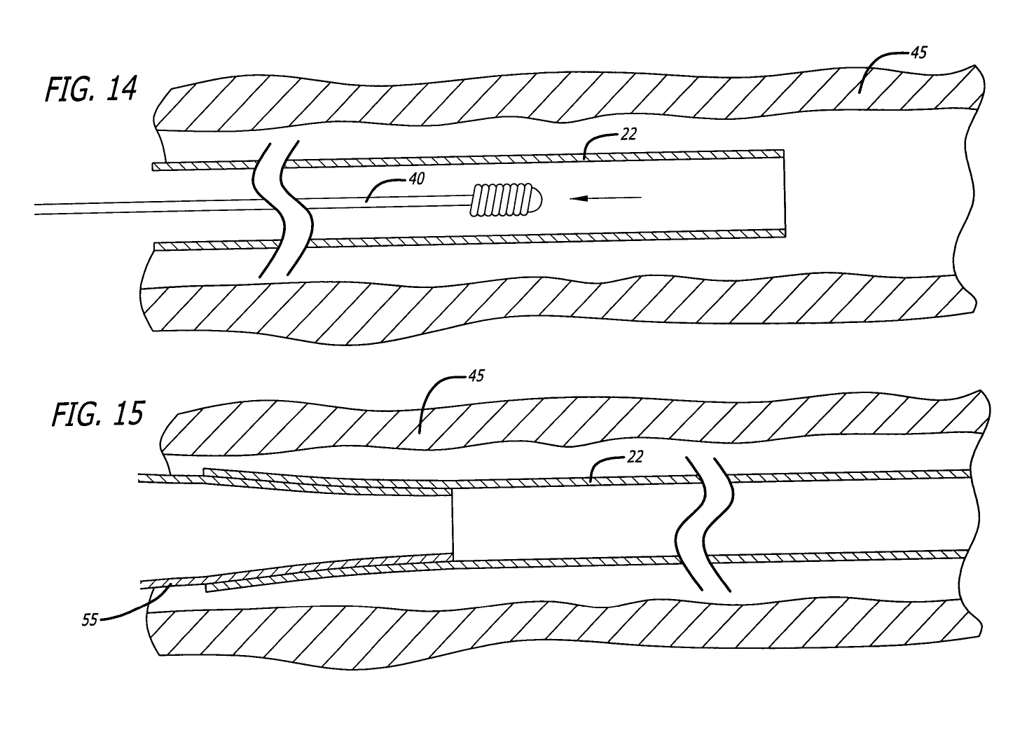

Once the guidewire 40 is properly positioned, the introducer assembly 20 may be inserted into the patient's vasculature 45 by threading the inner core 24 along the guidewire until the distal tip 26 of the inner core reaches the desired location with the vasculature (e.g., proximate the superior vena cava), as shown in FIG. 11. As shown in FIG. 12, once the distal portion of the introducer assembly is at the desired site in the patient's vasculature, the outer sheath 32 is withdrawn while the inner sheath 22 maintains its position as a result of the release mechanism (e.g., ripcord or suture) 28 removably affixing the inner sheath to the inner core's distal tip. After removal of the outer sheath, the ripcord may then be pulled to release the inner sheath from the distal tip. Upon release, the ripcord and the inner core are removed from the vasculature, as shown in FIG. 13. As shown in FIG. 14, the guidewire 40 may then be removed leaving only the inner sheath 22 in the vasculature. Alternatively, the guidewire may remain in the inner sheath so that the medical device may be steered over the guidewire during insertion through the inner sheath.

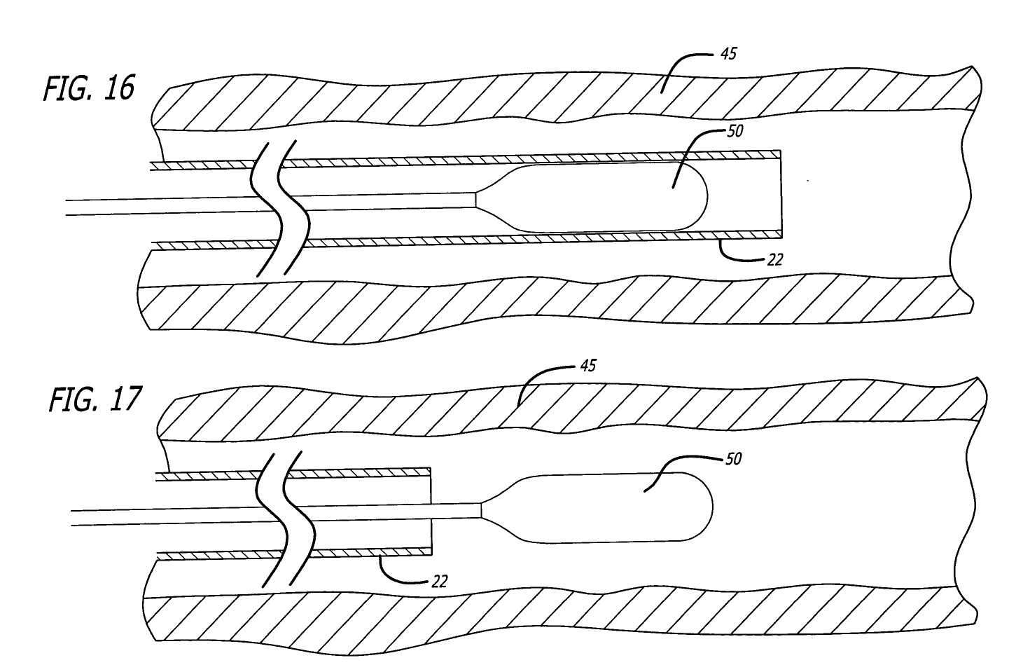

Upon removal of the guidewire 40, the inner sheath 22 is positioned and configured to introduce and guide a medical device (e.g., a percutaneous catheter) 50 into the body passageway 45. Prior to introducing the medical device and as shown in FIG. 15, a funnel-shaped dilator 55 (e.g., graduated from a size 20F to a size 32F) may be inserted into the inner sheath (removed from the handle 36) at the insertion site so as to expand the diameter of the proximal end of the inner sheath. Alternatively, the medical device may be inserted through the introducer handle (FIG. 7). The dilator may be removed after the distal portion of the medical device (catheter) is positioned within the proximal portion 23 of the inner sheath. As shown in FIG. 16, the distal portion of the medical device is advanced through the vasculature until the distal end of the device reaches the desired location (e.g., within the superior vena cava). Upon proper placement, the inner sheath may be withdrawn while leaving the medical device positioned within the patient for a time for the medical device to be effective. Alternatively, the inner sheath may be partially withdrawn while the medical device accomplishes its intended purpose (FIG. 17). After which the medical device may be retracted into the inner sheath so that the sheath and device are withdrawn together from the patient. Further, the medical device may be withdrawn first, followed by removal of the inner sheath, or visa versa.

While several particular forms of the invention have been illustrated and described, it will be apparent that various modifications can be made without departing from the spirit and scope of the invention. For example, references to materials of construction and specific dimensions are also not intended to be limiting in any manner and other materials and dimensions could be substituted and remain within the spirit and scope of the invention. Accordingly, it is not to be intended that the invention be limited, except as by the appended claims.

1. An introducer assembly, comprising:
an elongate tubular inner sheath formed of a retractable material;
an inner core removably positioned within the inner sheath; and
an elongate tubular outer sheath, wherein the inner sheath having the inner core disposed therein is removably positioned within the outer sheath.

2. The introducer assembly of claim 1, further including a releasing mechanism for removably attaching a distal portion of the inner sheath to a distal portion of the inner core.

3. The introducer assembly of claim 2, wherein the releasing mechanism includes a cord having a proximal portion disposed within the outer sheath and a distal portion configured to removably attach the inner sheath to the inner core.

4. The introducer assembly of claim 3, wherein the cord is at least partially disposed within a lumen of the inner core.

5. The introducer assembly of claim 4, wherein the inner core is formed with a distal tip having a distally descending conical tapered surface.

6. The introducer assembly of claim 5, further including a guidewire disposed within the lumen of the inner core.

7. The introducer assembly of claim 1, wherein the inner diameter of the outer sheath is smaller than the outer diameter of the inner sheath.

8. The introducer assembly of claim 7, wherein the outer sheath maintains the inner sheath in a folded state.

9. The introducer assembly of claim 1, wherein the outer sheath is formed from a flexible material.

10. The introducer assembly of claim 9, wherein the outer sheath is formed from a material that resists kinking.

11. The sheath assembly of claim 1, wherein the sheath assembly has a profile of about twenty French.

12. An assembly for positioning a medical device within a body passage, comprising:
inner core means for receiving a guidewire;
inner sheath means for receiving a medical device, the inner sheath means being formed from a retractable material and being removably disposed over the inner core means; and
outer sheath means for maintaining the inner sheath means disposed about the inner core means.

13. The assembly of claim 12, further including means for removably securing a distal portion of the inner sheath means to a distal portion of the inner core means.

14. The assembly of claim 13, further including means for attaching the means for removably securing to the inner core means.

15. An introducer, comprising:
a first sheath formed from a retractable material and configured to introduce a medical device into a vasculature of a patient;
a core member having a proximal section and a distal section and configured with a longitudinal lumen extending from the proximal section to the distal section, the core member being disposed within the first sheath;
a second sheath having a proximal section and a distal section and being formed from a flexible material, wherein the first sheath and the core member are disposed within the second sheath; and
a ripcord having a proximal section and a distal section and being configured to removably attach the first sheath to the core member.

16. The introducer of claim 15, wherein the proximal section of the ripcord is disposed between the first sheath and the second sheath, and wherein the distal section of the ripcord is wound about the distal section of the first sheath.

17. The introducer of claim 15, wherein the proximal section of the ripcord is disposed within the lumen of the core member, and wherein the distal section of the ripcord is tied to the distal section of the first sheath.

18. The introducer of claim 15, further comprising a guidewire disposed with the lumen of the core member, wherein the proximal section of the first sheath is configured with a handle adapted to receive the guidewire, wherein the lumen of the core member is adapted to receive the guidewire, and wherein the distal section of the core member is tapered.

19. A method for introducing a medical instrument into a vasculature, comprising:
providing an introducer assembly having
(a) inner core means for receiving a guidewire,
(b) inner sheath means for receiving a medical instrument, the inner sheath means being formed from a retractable material and being removably disposed over the inner core means,
(c) means for removably securing a distal portion of the inner sheath means to a distal portion of the inner core means, and
(d) outer sheath means for maintaining the inner sheath means disposed about the inner core means;
inserting a guidewire into a vasculature of a patient;
threading the introducer assembly over the guidewire and into the vasculature;
withdrawing the outer sheath means from the vasculature;
releasing the distal portion of the inner sheath means from the distal portion of the inner core means;
withdrawing the means for removably attaching from the vasculature;
withdrawing the inner core means from the vasculature;
inserting a medical instrument into the inner sheath means;
advancing the medical instrument within the inner sheath means and into the vasculature; and
retracting the inner sheath means from the vasculature.

20. The method of claim 19, wherein providing an introducer assembly includes securing the means for removably attaching to the inner core means, and releasing the distal portion of the inner sheath means includes moving the distal portion of the inner core means relative to the distal portion of the inner sheath means.
